(19) United States
(12) Patent Application Publication
(10) Pub. No.: US 2004/0186461 A1
(43) Pub. Date: Sep. 23, 2004

(54) Catheter with an adjustable cuff

(76) Inventor: Kristian DiMatteo, Watertown, MA (US)

Correspondence Address: FAY KAPLUN AND MARCIN LLP, 150 BROAD, SUITE 702, NEW YORK, NY, 10038, US

(21) Appl. No.: 10/390,315

(22) Filed: Mar. 17, 2003

(51) Int. Cl. A61M 25/16

(52) U.S. Cl. 604/539

(57) ABSTRACT

A locatable cuff for coupling with a complementary projection on a catheter, allowing secure placement of the cuff at different locations on the catheter. In one exemplary embodiment, the cuff comprises an internal receiving location to fit over and receive the complementary projection of the catheter. A physician can select the preferred location on a catheter for the cuff either before or during surgery.

### FIELD OF THE INVENTION

The invention relates generally to medical devices used to access portions of a body, such as vasculature.

### BACKGROUND OF THE INVENTION

Catheters are used in the health care industry to provide long-term vascular access, for administration of intravenous (IV) fluids, blood products, medications, parenteral nutrition solutions, blood withdrawal, and more. Such catheters generally have an elongated flexible body and include one or more lumens for the transfer of fluids. Catheters exhibiting these characteristics include thin-walled catheters, dialysis catheters, high-flow catheters, high-pressure catheters, and chronic tunneled central catheters. These can be percutaneously inserted into a body, sometimes using “tunneling” techniques. A subcutaneous tunnel or pocket is created, into which a catheter is inserted. After placement, the catheter extends from an access site (such as a selected vein), through the tunnel, to an exit site (e.g., on a patient's chest or arm). Tunnels can be formed in various sizes and configurations, to meet varying surgical requirements.

Some catheters have a polyester cuff fixedly disposed about a small portion of the elongated body. The cuff is surgically positioned near the underside of the cutaneous layer, to facilitate and promote tissue ingrowth. Tissue growth into the porous material of the cuff creates a barrier to infection, separating inner portions of the body from the incision site. The likelihood of infection into the body from the exit site of the catheter is thereby reduced. Fixation of the catheter in the subcutaneous tunnel is also enhanced by the presence of the cuff and the resulting tissue ingrowth.

Unfortunately, the various tunnel sizes and configurations used for catheter placement result in the need for catheters possessing different physical characteristics. For example, a short catheter used in a long tunnel results in positioning of the cuff too far beneath the cutaneous layer. Conversely, using a short tunnel with a long catheter results in extra catheter tubing within the tunnel when the cuff is properly positioned.

Surgical techniques and cuff placement preferences are physician specific. Cuff positioning within the tunnel varies by physician, requiring an inventory of catheter/cuff combinations to be maintained. Frequently a medical institution must inventory large numbers and various types of catheter/cuff combinations. This requirement can be somewhat mitigated for some types of catheters (e.g., PICC catheters) if physicians are willing to trim excessively long catheter lengths before surgery—but there are drawbacks. For example, trimming the end of the catheter can result in a tip with unwanted sharp edges, and the modified surface can hinder effective catheter placement at the access site. Furthermore, it is not always apparent until after commencement of the surgery what catheter length will be required between the catheter tip and the cuff. This issue can not be overcome by trimming the catheter length before surgery.

What is needed is a catheter with an adjustable cuff that accommodates the cuff placement preferences of different physicians. The cuff needs to be locatable and positionable on a catheter, preferably at different locations about the catheter. A physician needs to be able to conveniently position the cuff after surgery has commenced, and should not have to decide before surgery at what location on the catheter the cuff will be attached.

### SUMMARY OF THE INVENTION

In one aspect the invention features a catheter cuff positioning system comprising a catheter tube that includes an external mounting projection, and a locatable cuff adapted to mate with the external mounting projection. The external mounting projection can be integrally formed with the catheter tubing, and it can protrude out of or into the catheter tubing. Certain embodiments of the external mounting projection include a textured surface, while other embodiments of the catheter cuff positioning system can include a plurality of external mounting projections. The external mounting projections can have rounded edges, to name but one configuration. In certain embodiments, external mounting projections can be disposed about the catheter.

A cuff of the present invention can be disposed between two external mounting projections to establish a position of the cuff on the catheter. Alternatively, the cuff can have an internal receiving location adapted to mate with an external mounting projection. The internal receiving location can be adapted to fit over and receive any one of or a plurality of external mounting projections. In some embodiments the external mounting projections comprise a plurality of shapes. In one preferred embodiment, an external mounting projection includes a ridge disposed about a perimeter of the catheter tube, that is transverse to a longitudinal axis of the catheter tube. The external mounting projection can also comprise a shallow groove.

As contemplated herein, embodiments of the present invention include a catheter cuff positioning system wherein the catheter is a thin-walled catheter, a dialysis catheter, a high-flow catheter, a high-pressure catheter, and a chronic tunneled central catheter.

Another aspect of the invention features a catheter comprising a tube and an external mounting projection adapted to receive a locatable cuff. The external mounting projection can protrude relative to an exterior wall of the catheter tube. The external mounting projection can be integrally formed with the exterior wall of the catheter tube, and can protrude into or out of the catheter tube. The external mounting projection can include a textured surface.

Certain preferred embodiments of the catheter of the present invention include a plurality of external mounting projections. In certain other preferred embodiments, the catheter tube and the external mounting projection can be manufactured from different materials. In one exemplary embodiment, an external mounting projection can be fabricated from a material including at least one of polyurethane and silicone, and the catheter tube can be manufactured from a material including at least one of polyurethane and silicone. By way of example, the external mounting projection can be attached to the catheter tube, and it can be attached by at least one of an adhesive or a thermal joining technique, to name but a few.

Catheters of the present invention are selected from the group of thin-walled catheters, dialysis catheters, high-flow catheters, high-pressure catheters, and chronic tunneled central catheters. The skilled artisan will recognize other suitable catheter embodiments of the present invention.

Yet another aspect of the invention features a locatable cuff. Such a cuff can couple with a complementary projection on a surface of a catheter. In one embodiment, the locatable cuff includes an internal receiving location to receive a complementary projection of a catheter. By way of example, the internal receiving location can comprise a recess, and the recess can comprise a groove. By way of another example, the internal receiving location can include an interior surface textured to mate with an external textured surface of the catheter.

The locatable cuff can be manufactured from materials including Dacron® (registered trademark of E.I. du Pont de Nemours and Company, Wilmington, Del.), other polyesters, felts, and velours. In one preferred embodiment, it can comprise two fastenable halves and can be secured in a closed position using, for example, a biocompatible adhesive material.

### BRIEF DESCRIPTION OF THE DRAWINGS

These and other features of the invention are more fully described below in the detailed description and accompanying drawings, of which the figures illustrate an apparatus and method for adjusting the cuff position on a medical device such as a catheter.

FIGS. 1A and 1B illustrate exemplary embodiments of the inventions set forth herein.

FIGS. 2A and 2B each show a side view of exemplary catheter embodiments.

FIGS. 3A-E show cross-sectional views of different exemplary catheter embodiments.

FIGS. 4A-D illustrate exemplary embodiments of catheter exterior surfaces.

FIGS. 5A-E illustrate exemplary cuff embodiments.

FIGS. 6A-C are perspective views of exemplary embodiments of a cuff, illustrating one half of a cuff for each embodiment shown.

FIGS. 7A-F each show a side elevation view of a catheter and cuff joined together, representing only a few of the exemplary embodiments of the invention as contemplated herein.

FIGS. 8A and 8B each show a cross section of exemplary embodiments of a catheter and cuff joined together.

FIGS. 9A-D illustrate only a few of the ways in which portions of exemplary embodiments of the cuff can be fastened together as contemplated herein.

### DETAILED DESCRIPTION

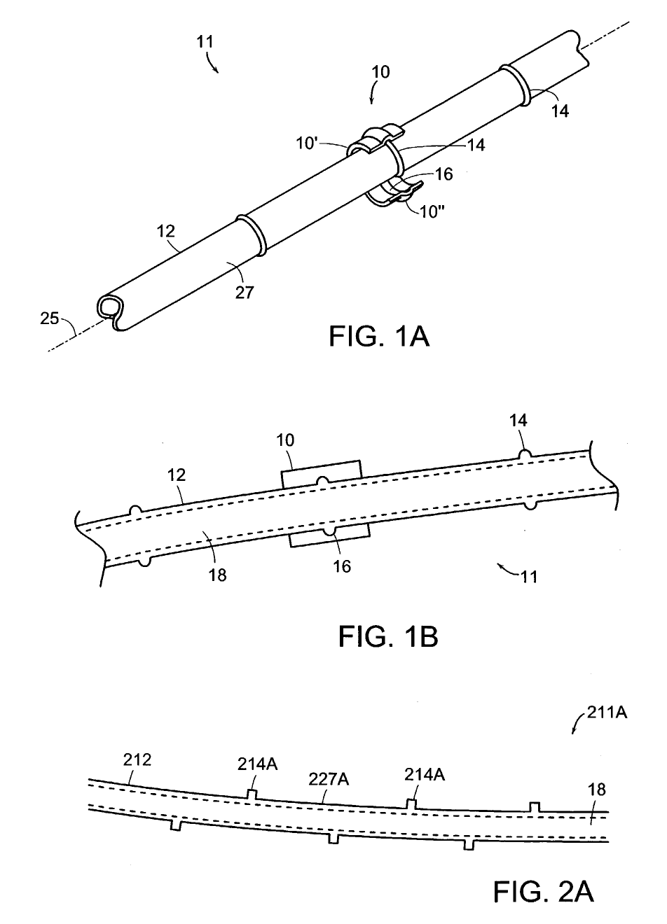

Referring to the drawings, FIGS. 1A and 1B illustrate an exemplary embodiment of the invention. A locatable cuff 10 is disposed about a catheter 11 comprising catheter tube 12 and an external mounting position 14. The external mounting position can include a projection such as a ridge, as shown in FIG. 1A. In this embodiment, the cuff 10 includes an internal receiving location 16 to receive the complementary projection of the external mounting position 14.

FIG. 1A illustrates a cuff 10 comprising two halves 10′ and 10″. The two cuff halves 10A, 10B can be closed about the external mounting position 14, thus securing the position of the cuff with respect to the catheter tube 12. The catheter has a length along catheter axis 25 and the external mounting positions can be disposed along this length, as shown. The catheter tube 12 has an exterior surface 27. External mounting positions 14 are adjacent the exterior surface 27 of the catheter. FIG. 1B shows a side view of a cuff 10 attached to a catheter tube 12 about external mounting position 14. The catheter also includes lumen 18, the boundaries of which are represented in the figure by dashed lines. The invention contemplates other embodiments, examples of which are discussed below.

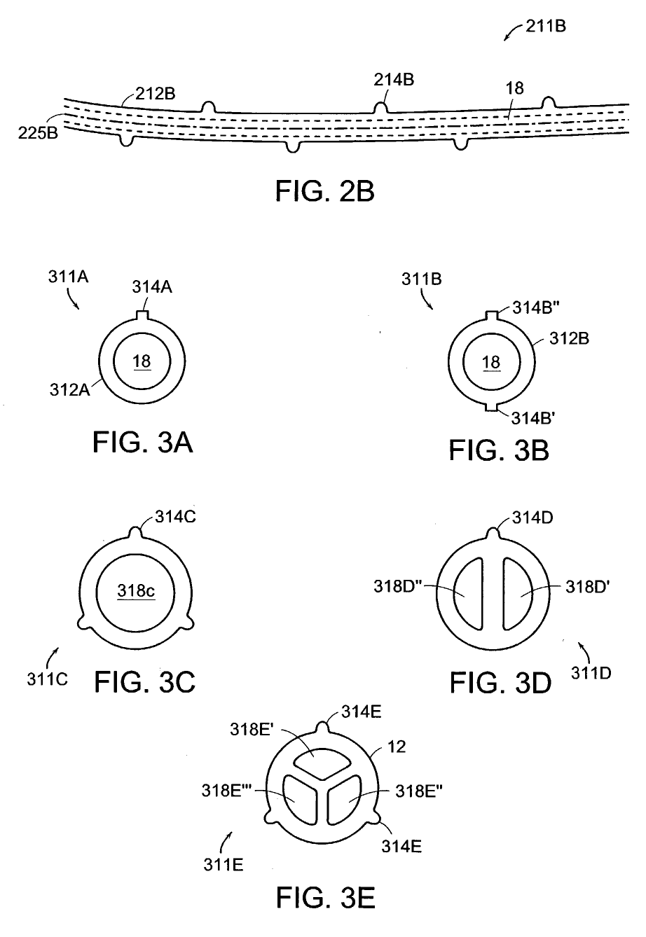

FIGS. 2A and 2B each show a side view of different catheter embodiments. These figures show that the catheter tube 12 can include external mounting positions disposed at various points about the catheter. External mounting positions 14 (e.g., 214A) are shown spaced along a length of the catheter. Although the external mounting positions 214A of FIG. 2A are uniformly spaced along a portion of the length of the catheter, this is not necessary in all embodiments. The external mounting positions 14 can protrude from the catheter at discreet locations. These discreet locations can be at different points along the length of the catheter tube 12, and they can also be disposed at different points about the circumference.

External mounting positions (e.g., 214A) can have abrupt, square edges. Other embodiments include external mounting positions with rounded edges and surfaces, such as external mounting positions 214B of FIG. 2B. Alternatively, a single protrusion of an external mounting position can comprise both square and smooth, rounded edges and surfaces. Other embodiments can include other shapes. For example, external mounting positions can have an oval, rectangular, or star shape. Moreover, a single external mounting position can include multiple projections, discussed below.

Catheters (e.g. 211A, 211B) such as thin-walled catheter, dialysis catheters, high-flow catheters, and high-pressure catheters generally include at least one lumen (e.g., 218A, 218B). For example, thin-walled embodiments can have wall thicknesses of only 0.010 to 0.015 inches. The unobstructed flow of fluid through the lumen in such catheters can be an important catheter design and selection parameter (e.g., 218A, 218B).

FIGS. 3A-E show cross-sectional views of different catheter embodiments. FIG. 3A shows a cross-sectional view of catheter 11 (e.g., 311) according to an embodiment of the invention, comprising external mounting position 314A with square edges. In this depiction, only one external mounting position is disposed about the circumference of catheter tube 312, although multiple external mounting positions can be disposed along its length. FIG. 3B is an exemplary embodiment with two external mounting positions disposed about the circumference of catheter tube 312B. Multiple external mounting positions 314B′ and 314B″ can be disposed along the length of catheter 12. The particular locations of external mounting positions along the length of catheter 12 can be spaced regularly or irregularly. For example, external mounting positions 314B′ and 314B″ can be disposed symmetrically across from each other, or they can be in a staggered arrangement. Alternatively, they can be positioned irregularly along the length of the catheter (e.g., 311A, 311B).

FIG. 3C is another embodiment comprising three external mounting positions about the circumference of the catheter. These external mounting positions are shown with rounded surfaces. The catheter of this exemplary embodiment has only one lumen 318C, however, a plurality of lumens is also contemplated herein. Any of the embodiments shown in FIGS. 3C-E can include external mounting positions with rounded and/or square edges and surfaces. They can also have one, two, three, or more external mounting positions disposed at different points about a circumference of catheter tube 12, and one or a plurality of external mounting positions disposed along the length of the catheter.

Catheters with different numbers of lumens are used for different purposes, as is known to the skilled artisan. For example, the catheter of FIG. 3D includes two lumens, 318D′ and 318D″, while catheter 311E comprises three lumens (318E′, 318E″, and 318E′″). Moreover, the cross-sectional lumen shapes illustrated are exemplary only. Embodiments of the present invention include other lumen configurations and cross sections, such as multiple lumens of different sizes. The different lumens can also have different cross-sectional shapes. An embodiment (not shown) includes one round and two oval lumens.

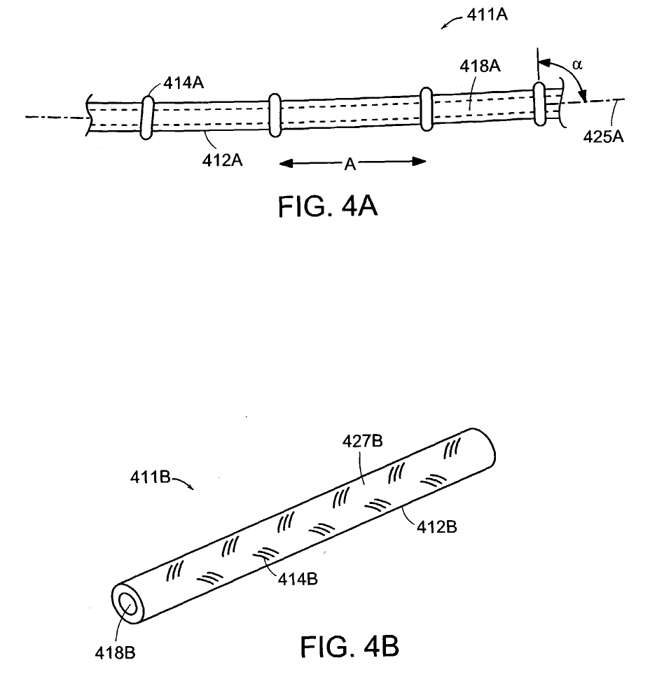

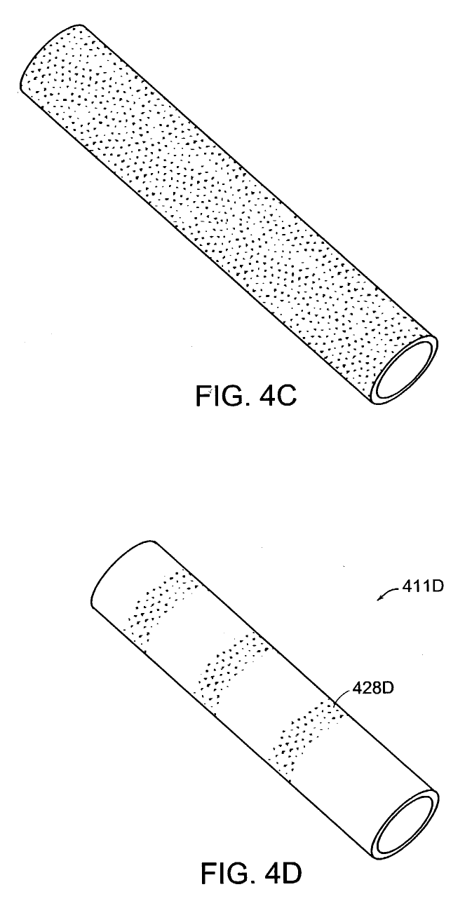

FIGS. 4A-D illustrate different exemplary catheter exterior surface embodiments. Embodiments include various exterior surfaces, such as catheters comprising external mounting positions including external ridges (e.g., FIG. 4A). In this embodiment the ridges extend entirely around the circumference of the catheter tube 12. As shown, external mounting positions 414A are evenly spaced along a portion of catheter 411A, along a length that extends in parallel with catheter axis 425A. Even spacing of external mounting positions 414A is not required in all embodiments. The external mounting positions can be located along the entire length of catheter tube 412, or along only a portion of the length.

As shown, external mounting position 414A can be shaped as a ridge disposed about a perimeter of the catheter tube, which is oriented in a direction transverse to longitudinal catheter axis 425A. Different angles of orientation (α) can also be used. The external mounting positions 14 (e.g., 414A) can be integrally formed with the catheter tube, or they can be manufactured separately and attached to it. The external mounting positions do not protrude into the lumen 418A of catheter 411A.

FIG. 4B illustrates a catheter 411B in which external mounting position 414B is adjacent the exterior surface 427B of catheter tube 412B and comprises a textured surface. In certain embodiments, the textured surface of the external mounting position 14 (e.g., 414B) can include various slotted patterns (e.g., FIG. 4B) that are disposed over all or a portion of the exterior surface 27 (e.g., 427B) of the catheter tube 12 (e.g., 412B). The textured pattern can also include dimples or other protrusions (FIG. 4C), and they can be disposed in patterns such as bands 428D about the catheter (e.g., 411D). As in other embodiments, an external mounting position comprising textured surfaces can be present alone or in combination with one or more other external mounting positions.

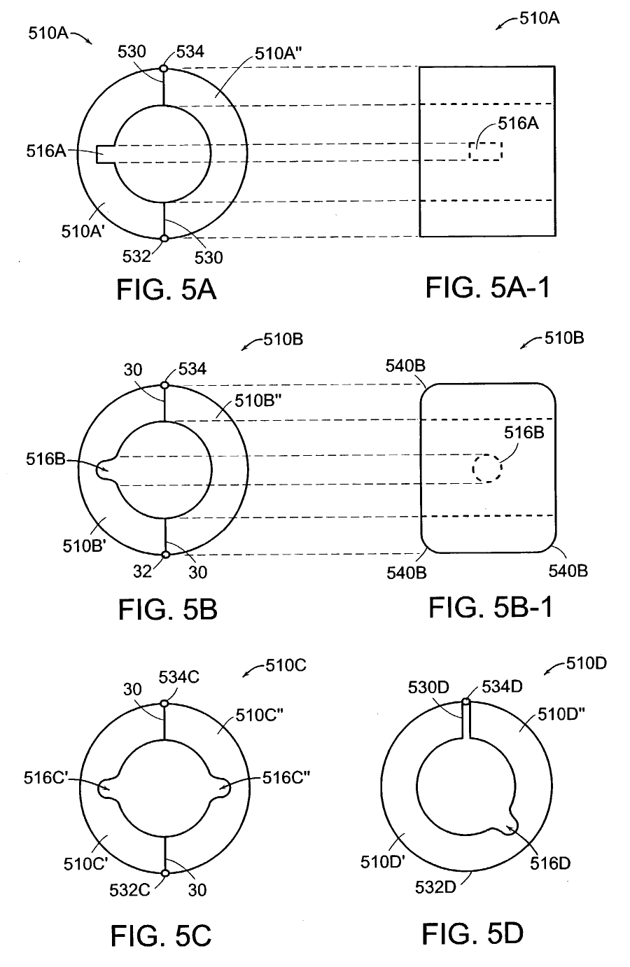

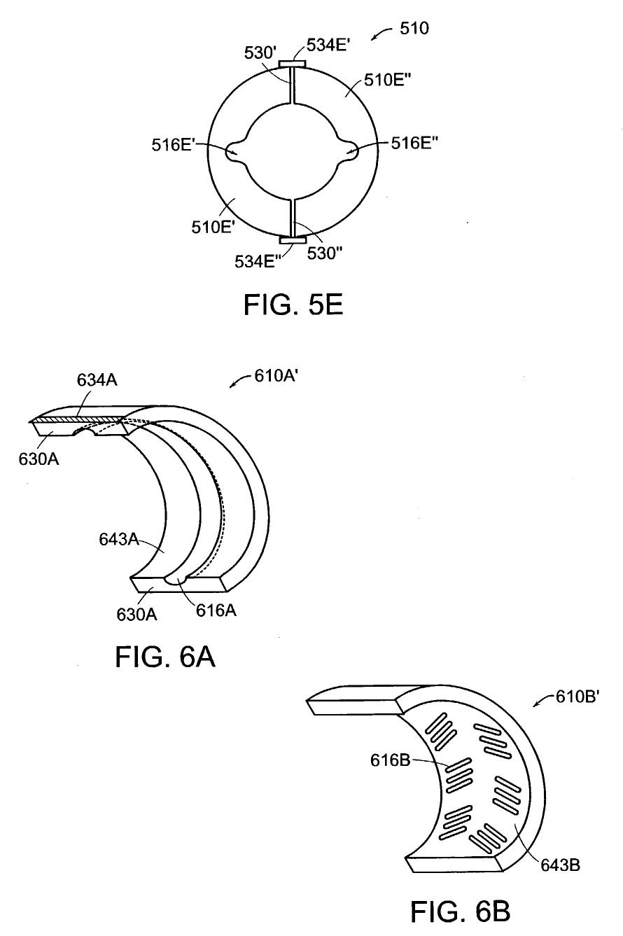

FIGS. 5A-E illustrate different exemplary cuff embodiments of the invention.

FIG. 5A shows an internal receiving location 516A disposed within a first cuff half 10′ (i.e., 510A′). First cuff half 510A′ can mate with second cuff half 510A″ to form cuff 510A. The two cuff halves 510A′ and 510A″ contact each other at cuff mating surfaces 30 (e.g., 530). Cuff mating surfaces 530 can include a cuff joint 32 (e.g., 532) and a cuff fastener 34 (e.g., 534). Cuff joint 532 acts as a hinge mechanism that can allow the cuff mating surfaces 530 at the opposite side of the cuff to be separated, placing the cuff in an open position. When the cuff halves are separated and the cuff is in an open position, the cuff can be positioned over a catheter tube 12. After placement about a portion of a catheter the two cuff halves (e.g., 510A′ and 510A″) can be placed together and the cuff fastener can be used to secure the two cuff halves in a closed position.

As shown in FIG. 5A, the second cuff half 510A″ does not have an internal receiving location. In other embodiments, both cuff halves have internal receiving locations. The internal receiving location 516A of the first cuff half 510A′ has square edges, which can mate with the square edges of an external mounting position (e.g., 214A). Internal receiving location 16 (e.g., 516A) can fit over and receive any of the external mounting positions 214A of catheter 211A (FIG. 2A). External mounting position 214A protrudes from the exterior wall 227A of the catheter 211A. The internal receiving location 514A can include a recess that receives external mounting position 214A. Thus, cuff 510A is an embodiment of a locatable cuff that can be coupled with a complementary projection (e.g., external mounting position 14, such as 214A) on a surface of a catheter (e.g., 227A).

When external mounting position 14 is positioned within internal receiving location (e.g., 516A) and the cuff halves 10′ and 10″ are fastened using cuff fastener 34, placement of the cuff 10 (e.g., 510A) on the catheter is secured and motion of the cuff 10 with respect to the catheter is restricted. In this embodiment, the cuff is secured with respect to the catheter in either a longitudinal or a circumferential direction.

FIG. 5B shows another cuff embodiment, in which internal receiving location 516B comprises rounded edges. The rounded edges of internal receiving location 16 (e.g., 516B) can efficiently mate with external mounting positions such as external mounting positions 214B of FIG. 2B. The two cuff halves 10′ and 10″ (e.g., 510B′and 510B″) can join with each other to form cuff 510B, contacting each other at cuff mating surfaces 30. Cuff 510B includes a cuff joint 32 and cuff fastener 34. The cuff halves 510B′ and 510B″ can be separated and the cuff can be placed about a catheter 12. The internal receiving location 516B can fit over and mate with a complementary protrusion, such as an external mounting position 14. The cuff halves can be secured in a closed position using fastener 34 (e.g., 534B), such that cuff mating surfaces 30 contact each other.

The internal receiving location 516B of cuff 510B has rounded edges, which are internal to the cuff. Use of rounded edges and surfaces within the cuff complements the usage of external mounting positions with rounded surfaces. Additionally, smooth and rounded surfaces can be used on the exterior surfaces of the cuff, such as rounded edges 540B. When internal receiving location 16 (e.g., 516B) fits over and receives external mounting position 14, movement of the cuff about the catheter in both longitudinal and circumferential directions is restricted.

A cuff embodiment including two internal receiving locations 516C′ and 516C″ is shown in FIG. 5C. The two cuff halves 510C′ and 510C″ can join with each other to form cuff 510C, contacting each other at cuff mating surfaces 530. Cuff 510C includes a cuff joint 532 and cuff fastener 534. The cuff halves 510C′ and 510C″ can be separated and the cuff can be placed about a catheter 12. The internal receiving locations 516C′ and 516C″ can fit over and mate with complementary protrusions, such as the external mounting position 214B. In this embodiment, the cuff would have to be sufficiently wide to encompass at least two external mounting positions 214B (FIG. 2B), since the external mounting positions are in a staggered arrangement and are not located symmetrically with respect to each other about the catheter axis 225B. The cuff halves 510C′ and 510C″ can be secured in a closed position about the catheter using fastener 534C, such that cuff mating surfaces 530C contact each other and external mounting positions 214C are located in internal receiving locations 516C′ and 516C″.

Another cuff embodiment 510D is shown in FIG. 5D, comprising cuff fastener 534D and cuff joint 532D. In this embodiment, the hinge mechanism of cuff joint 532D comprises a resilient portion of the cuff 510D and no distinct hinge mechanism is present or required. The two cuff halves 510D′ and 510D″ can join with each other to form cuff 510D, contacting each other at cuff mating surfaces 530D and encircling the catheter tube 12. The cuff halves 510D′ and 510D′ can be separated and the cuff can be placed about a catheter 12. The internal receiving location 516D can fit over and mate with a complementary protrusion, such as an external mounting position 14. The cuff halves can be secured in a closed position using fastener 534D, such that cuff mating surfaces 530D contact each other. When internal receiving location 516D fits over and receives external mounting position 14, movement of the cuff about the catheter in both longitudinal and circumferential directions is restricted.

Other cuff embodiments do not have a cuff joint 34, but instead have two or more cuff fasteners. An exemplary embodiment is illustrated in FIG. 5E. This embodiment includes cuff fasteners 534E′ and 534E″. Both cuff halves (e.g., 510E′ and 50E″) can be secured to each other using the cuff fasteners, such that mating surfaces 530′ and 530″ contact each other. One or both internal receiving locations (516E′, 516E″) can mate with an external mounting position 14 of the catheter tube 12. Other embodiments include resilient cuffs that can be stretched to fit over the catheter tube 12, and which require no fastener.

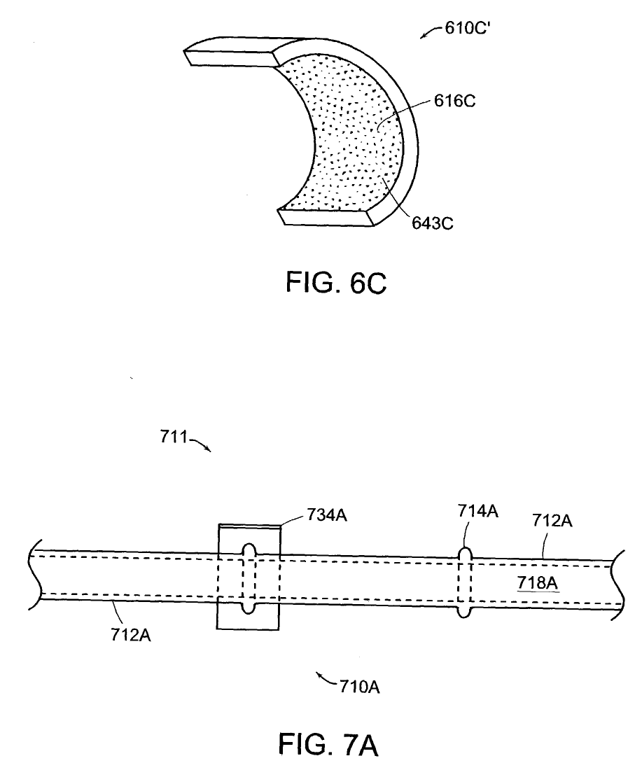

FIGS. 6A-C are perspective views of different exemplary embodiments of a cuff 10, illustrating one half of a cuff for each exemplary embodiment shown. FIG. 6A shows a cuff half 610A′ in which internal receiving location 616A comprises a groove. The groove is formed in an internal surface 643A of the cuff and is adapted to fit over and receive embodiments of the external mounting position (e.g., 414A, FIG. 4A). The position of the cuff 610A on a catheter 412A can be fixedly established by securing cuff 610A over external mounting position 414A, thus preventing movement of the cuff along the catheter. The cuff can be secured using a cuff fastener, described in more detail below.

FIG. 6B shows an embodiment of a cuff half 610B′ in which the interior surface 643B comprises an internal receiving location 616B including a textured surface. This textured surface mates with a corresponding surface, such as a textured surface of an external mounting position 414B, thus facilitating the securement, placement, and positioning of the cuff on the catheter. FIG. 6C illustrates a different type of textured surface that can be used with a cuff. In this embodiment, the interior surface 643C of the cuff half 610C′ comprises internal receiving location 616C including the textured surface shown. This surface has a plurality of bumps forming a continuous pattern about the interior surface 643C. In some embodiments, the textured surface can cover only portions of the interior surface 643C.

The internal receiving locations of each of these exemplary cuff embodiments (e.g., 610A-C) of internal receiving locations 616A-C has been described as mating with a corresponding structure of an external mounting position. However, features of these embodiments can be combined and interchanged. For example, cuff 610C can be fit over and receive external mounting position 414B and result in effective utilization of the invention, or cuff 610A can be used with external mounting position 414B. Similarly, cuff 610A can be used with external mounting position 214A if the corresponding structures are properly sized.

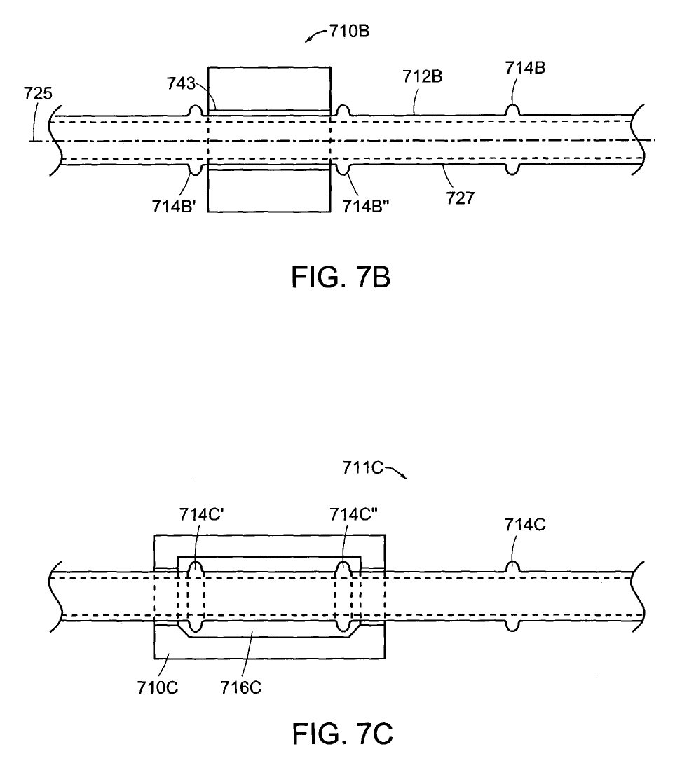

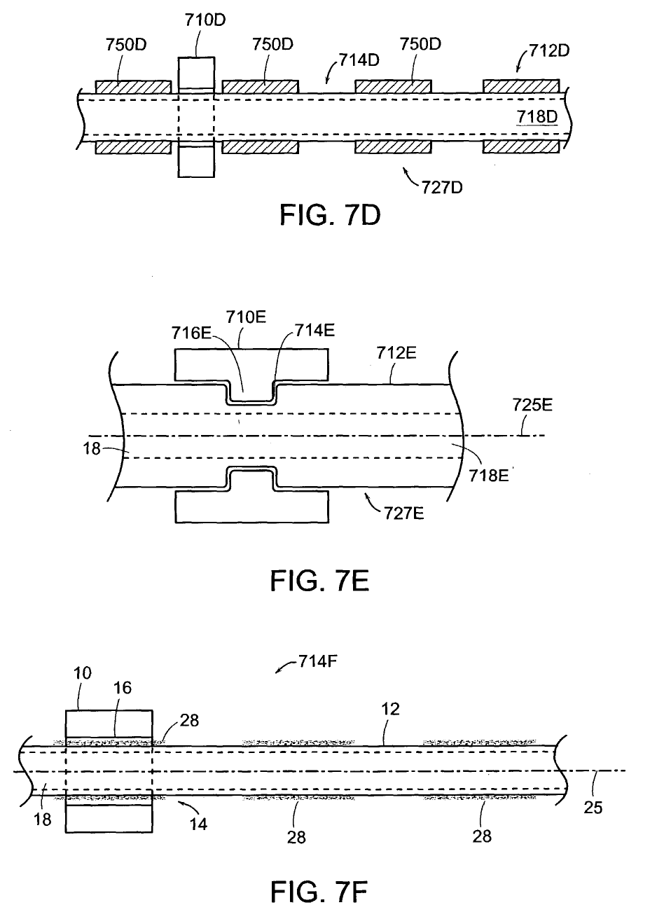

FIGS. 7A-F show a side elevation view of different catheter cuff positioning system embodiments. FIG. 7A illustrates a cuff 710A positioned and secured over external mounting position 714A. The cuff is fixedly secured to the catheter since the cuff fastener 734A secures together the two halves (not shown) of the cuff. In this embodiment, the external mounting positions 714A comprises a ridge extending about the circumference of catheter tube 712A. The external mounting positions are spaced along a portion of the catheter and no obstruction is present in lumen 718A.

FIG. 7B illustrates an embodiment in which the cuff 710B is positioned between and secured by two external mounting positions 714B. In this embodiment, longitudinal movement of the cuff along the catheter axis 725 is prevented by the external mounting positions. These external mounting positions are either integrally formed with the catheter tube 712 or are fixedly attached to it. The internal surface 743 of the cuff and/or the exterior surface 727 of the catheter tube can have textured surfaces, to facilitate mating between the internal receiving location 716B of the cuff and the external mounting position 714B of the catheter. In such embodiments, the surface of the catheter tube mating with the internal receiving location of the cuff, between the external mounting positions 714B′ and 714B″ can comprise a textured surface.

FIG. 7C shows internal receiving location 716C of cuff 710C as it fits over and receives two external mounting positions (e.g., 714C′ and 714C″) of catheter 711C. The external mounting positions 714C of this embodiment comprise ridges and rounded surfaces. In other embodiments, the external mounting positions do not comprise ridges or rounded edges. The width of internal receiving location 716C as shown is wide enough to receive two external mounting positions. Embodiments include internal receiving locations with widths that are wider.

FIGS. 7D and 7E illustrate embodiments in which the external mounting positions protrude into the catheter tube 712D. Cuff 710D can encircle the catheter tube and fit within the external mounting position 714D. External mounting position 714D secures the positioning of the cuff relative to the catheter. In some embodiments, it is important that the external mounting position not project into the lumen (e.g., 718D) of the catheter. Such a projection would provide an obstruction to fluid flow within the catheter, providing a hindrance to effective catheter operation. The same considerations apply to multi-lumen catheters.

The external mounting projection 714D of FIG. 7D is illustrated with a circular external configuration. Other embodiments include alternative shapes. For example, the external configuration can be square, hexagonal, octagonal, etc. It can also be keyed, or have an irregular shape. Non-circular external configurations of the external mounting projections are useful, for example, to prevent rotation of the cuff about the catheter.

To accommodate these considerations, embodiments include external mounting positions 14 that protrude into the exterior surface of the catheter 12 only a very small amount. Thus, embodiments comprise external mounting projections that form only a shallow groove about the circumference of the catheter tube. Other embodiments feature catheter tubes including extended regions (e.g., 750D) that in effect act as a thicker catheter tube. The cuff 10 (e.g., 710D) can then be secured by placement between these extended regions.

Another catheter cuff system embodiment comprises external mounting positions extending into the catheter tube 712E as illustrated in FIG. 7E. The cuff 710E in this embodiment is wider than the external mounting projection 714E. The extra width of the cuff extends along the catheter tube 712E along the exterior surface 727E in directions parallel to catheter axis 725E. As in other embodiments, the internal receiving location 716E mates with the external mounting projection 714E, securing placement of the cuff 710 with respect to the catheter 12 (e.g., 712E). As indicated above, embodiments include external mounting projections with non-circular external configurations.

Yet another embodiment of a catheter cuff positioning system is shown in FIG. 7F. The external mounting projections 14 (e.g., 714F) comprise bands 28 (e.g., 718F) comprising a textured surface (as shown) disposed along catheter tube 12. The cuff 10 can be positioned over the bands such that the internal receiving location 16 fits over and receives the external mounting projections, thus securing placement of the cuff with respect to the catheter. Bands 28 can be located at various positions along the length of the catheter. A plurality of bands can be included as shown. Embodiments can include only one band, or alternatively can include a plurality of bands in various arrangements.

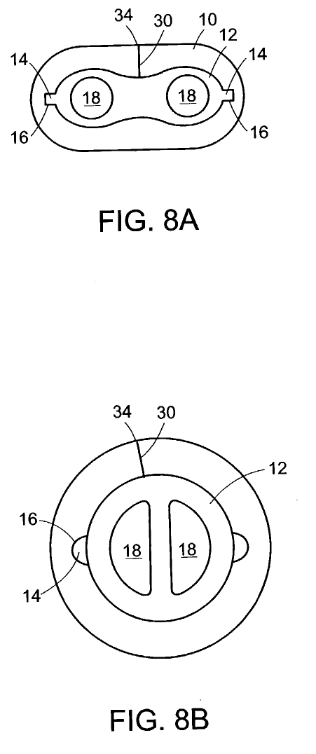

FIGS. 8A and 8B each show a cross-section of exemplary embodiments of catheter cuff positioning systems, in which a catheter and cuff are joined together. Cuff 10 surrounds catheter 12. External mounting projections 14 mate with internal receiving locations 16 and secure placement of the cuff with respect to the catheter. FIG. 8A shows a generally oval catheter cross-section, and FIG. 8B shows a generally circular catheter cross-section. Embodiments include other cross-sectional shapes. Each system shown (FIGS. 8A and 8B) include two lumens 18. Embodiments with two lumens can be used, for example, for dialysis. Other embodiments have different numbers of lumens. Various combinations of external mounting projections and internal receiving locations with square and rounded edges can also be used.

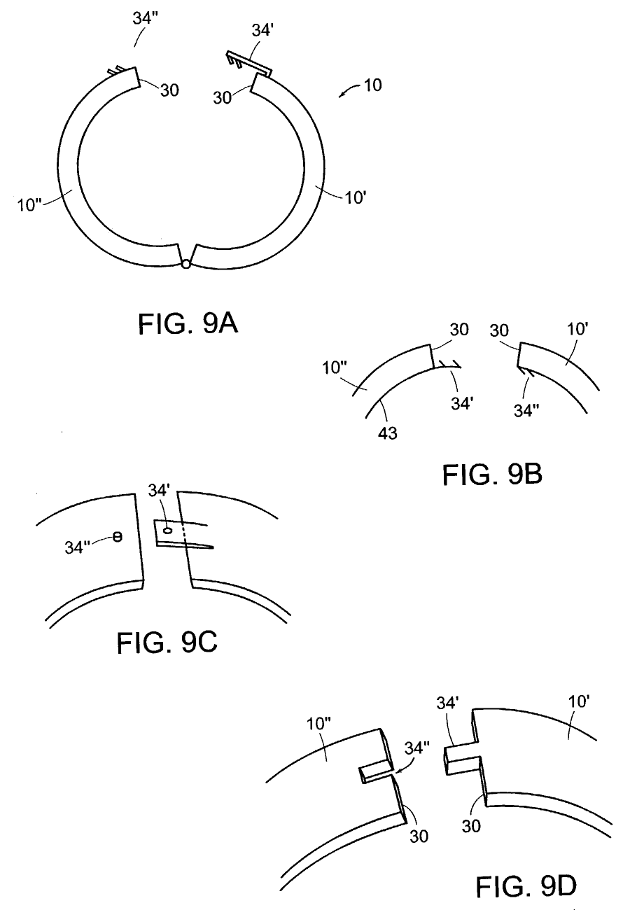

FIGS. 9A-D illustrate how portions of exemplary embodiments of the cuff can be fastened together. FIG. 9A illustrates a cuff fastener first member 34′ that fits over a second member 34″ external to the cuff 10. When in contact, the two fastener members prevent the two cuff halves 10′ and 10″ from being separated. In some embodiments the fastener is releasably secured. FIG. 9B shows the fastener members disposed on the inside surface 43 of the cuff 10.

Another fastener embodiment is illustrated in FIG. 9C. Fastener first member 34′ can be placed over fastener second member 34″ such that the second member fits within the first member and secures the fastener 34. FIG. 9D illustrates yet another embodiment of fastener 34. Member 34′ fits within member 34″ as the cuff halves 10′ and 10″ are closed together. Other embodiments (not shown) include other joining techniques and methods such as dadoes, dovetails, dowels, mortising, and combinations thereof.

The cuff halves can be secured together by a friction fit between fastener members 34′ and 34″. Embodiments include using a biocompatible adhesive to secure the cuff halves together. The adhesive can be applied to the cuff halves (10′ and/or 10″), the fastener halves (34′ and/or 34″), and the like. All cuff embodiments comprising cuff mating surfaces 30 can use adhesives and/or a mechanical snap fit.

External mounting projections 14 of the invention can be integrally formed with the catheter tube 12 (e.g., by molding), or they can be formed separately and attached. The catheter tube and the external mounting projection are formed of the same material when the external mounting projection is integrally formed with the catheter tube. Suitable materials include polyurethane, silicone, Teflon®, and other plastics. Suitable polyurethanes include polycarbonate-based polyurethanes, aromatic polyurethanes, and silicone polycarbonate-based polyurethanes. For example, a suitable aromatic polyether-based polyurethane is Tecothane® (registered trademark of Thermedics, Inc., Woburn, Mass.). A suitable aliphatic polycarbonate-based polyurethane is Carbothane® (registered trademark of Thermedics, Inc., Woburn, Mass.). When the external mounting projection is separately manufactured, forming the external mounting projection and the catheter tube of the same material reduces separation that can be caused by thermal separation effects.

However, in some embodiments the catheter tube and the external mounting projection are made from different materials. In these embodiments, the catheter tube can be formed of materials including polyurethane, silicone, Teflon®, and other plastics. Suitable polyurethanes include polycarbonate-based polyurethanes and aromatic polyurethanes. Embodiments of the external mounting projection include manufacturing it from materials including polyurethane, silicone, Teflon®, and other plastics. Suitable polyurethanes include polycarbonate based polyurethanes and aromatic or aliphatic polyurethanes. Solvent bonding, thermal forming, and other bonding techniques known to the skilled artisan can be used to attach external mounting projections to the catheter tube.

Other embodiments of the present invention include configurations for securing the cuff between external mounting projections. These external mounting projections can be spaced at 1 centimeter intervals, and can result in corresponding available adjacent cuff placement positions that are 1 centimeter apart. Cuff 10 (e.g., FIG. 7B) can then be secured between such external mounting projections using a snap fit, which can be provided by cuff fastener 34 (e.g., FIGS. 9A-C). Other spatial arrangements can also be used.

Other embodiments include a catheter cuff positioning system in which the external mounting projections of the catheter are designed to match, at least in part, corresponding internal receiving locations of the cuffs. While these features can be complementary, the degree of correspondence required between them need not be perfect and can be minimal. Only sufficient mating is required so as to prevent movement of the cuff with respect to the catheter, after the cuff has been securely placed and positioned. Thus, there can be a great deal of overlap between the interchangeability and inter-compatibility of the various cuff and catheter embodiments. For example, textured surfaces can be mated with those comprising discreet protrusions. Combinations that are functional are within the scope of the present invention if cuff placement is restricted relative to the catheter tube.

In other embodiments, permanent cuff attachment can be achieved using solvents, biocompatible adhesives, and the like (“adhesives”). Such adhesives can be applied to mating surfaces 30 to secure the cuff 10 in a closed position, e.g., about the catheter tube 12. They can also be applied to fastener 34 to achieve the same purpose. In some embodiments, adhesives can be applied, for example, between the interior surface 43 of the cuff and a portion of the catheter 11, to facilitate secure placement of the cuff 10. Adhesives can be pressure sensitive. For example, adhesive nodules can be placed on the interior surface 43 of the cuff, which can help to bind the cuff to the catheter when pressure is applied to the cuff and they are ruptured. Chemical bonding can also be used to bind the cuff to the catheter. Bonding agents can be applied to the interior surface 43 of the cuff, and/or to the exterior surface 27 of the catheter, prior to joining the cuff 10 and catheter together.

Similarly, adhesives and bonding techniques can be used to attach textured surfaces (described above) to the external mounting projections 14 and to the interior surface 43 of internal receiving locations 16 of the cuff 10. Textured surfaces can also be molded, integrally formed, and/or otherwise added to surfaces such as these.

Some embodiments include non-permanent attachment techniques for securing the locatable cuff 10 to the catheter 11. Mechanical attachment methods can be used for this purpose, and many fastener embodiments are available and known to those of skill in the art. Frictional and/or mechanical snap fit attachment methods and devices are also suitable, and can be used with the invention.

The cuff 10 can be fabricated from materials including Dacron® and polyester. Other suitable materials easily recognized as suitable by the skilled artisan can also be used. Cuff materials are preferably porous and allow the ingrowth of adjacent tissue. When a physician properly places the cuff in a body such ingrowth can create a barrier to infection, separating inner portions of the body from the incision site.

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A catheter cuff positioning system comprising:
a catheter tube comprising an external mounting projection; and
a locatable cuff adapted to mate with the external mounting projection.

2. The catheter cuff positioning system of claim 1 wherein the external mounting projection is integrally formed with the catheter tube.

3. The catheter cuff positioning system of claim 1 wherein the external mounting projection protrudes into the catheter tube.

4. The catheter cuff positioning system of claim 1 wherein the external mounting projection comprises a textured surface.

5. The catheter cuff positioning system of claim 1 comprising a plurality of external mounting projections.

6. The catheter cuff positioning system of claim 5 wherein the external mounting projections are disposed about the catheter tube.

7. The catheter cuff positioning system of claim 1 wherein the cuff is disposed between two external mounting projections to establish a position of the cuff on the catheter tube.

8. The catheter cuff positioning system of claim 1 wherein the cuff has an internal receiving location adapted to mate with the external mounting projection.

9. The catheter cuff positioning system of claim 6 wherein the cuff has an internal receiving location adapted to fit over and receive any of the plurality of external mounting projections.

10. The catheter cuff positioning system of claim 5 wherein the external mounting projections comprise a plurality of shapes.

11. The catheter cuff positioning system of claim 1 wherein the external mounting projection comprises a ridge disposed about a perimeter of the catheter tube that is transverse to a longitudinal axis of the catheter.

12. The catheter cuff positioning system of claim 1 wherein the catheter tube is selected from the group consisting of thin-walled catheters, dialysis catheters, high-flow catheters, high-pressure catheters, chronic tunneled central catheters, and PICC catheters.

13. A catheter comprising a tube and an external mounting projection adapted to receive a locatable cuff, the external mounting projection protruding relative to an exterior wall of the catheter tube.

14. The catheter of claim 13 wherein the external mounting attachment is integrally formed with the exterior wall of the catheter tube.

15. The catheter of claim 13 wherein the external mounting projection protrudes into the catheter tube.

16. The catheter of claim 13 wherein the external mounting projection comprises a textured surface.

17. The catheter of claim 13 further comprising a plurality of external mounting projections.

18. The catheter of claim 13 wherein the catheter tube and external mounting projection are manufactured from different materials.

19. The catheter of claim 13 wherein the external mounting projection is attached to the catheter tube.

20. The catheter of claim 18 wherein the external mounting projection is attached to the catheter tube by at least one of an adhesive and thermal joining techniques.

21. A locatable cuff for coupling with a complementary projection on a surface of a catheter, the locatable cuff comprising an internal receiving location to receive the complementary projection of the catheter.

22. The locatable cuff of claim 21 wherein the internal receiving location comprises a recess.

23. The locatable cuff of claim 22 wherein the recess comprises a groove.

24. The locatable cuff of claim 21 wherein the internal receiving location comprises an interior surface textured to mate with an external textured surface of the catheter.

25. The locatable cuff of claim 21 wherein the cuff comprises two fastenable halves.
